(19) United States
(12) Patent Application Publication
(10) Pub. No.: US 2021/0077081 A1
(43) Pub. Date: Mar. 18, 2021

(54) Rotary Oblique-Cutting Biopsy Needle

(71) Applicant: Harbin University of Science and Technology, Harbin (CN)

(72) Inventors: Yongde Zhang, Harbin (CN); Jiawei Zhao, Harbin (CN); Yifeng Hu, Harbin (CN); Zixi Zhao, Harbin (CN)

(21) Appl. No.: 17/016,529

(22) Filed: Sep. 10, 2020

(30) Foreign Application Priority Data
Sep. 17, 2019 (CN) 201910875547.9

(51) Int. Cl. A61B 10/02

(52) U.S. Cl. CPC A61B 10/0275; A61B 2010/0208

(57) ABSTRACT

A rotary oblique-cutting biopsy needle includes an internal needle and an external needle, where the internal needle and the external needle are in coaxial clearance fit; and an internal needle front end face is in contact with the external needle inner end face to guarantee that an oblique cutting edge and a cutting slot are longitudinally located at the same position. The external needle is fixed after the external needle and the internal needle are inserted into a human body together, and the internal needle axially rotates to cut lesion tissue. Axial rotary cut-off can effectively reduce errors caused by longitudinal displacement of the biopsy needle, and an internal needle oblique cutting edge has an inclination of 30°, the reliability of the tissue cutting process is improved

### RELATED APPLICATIONS

This application claims priority to Chinese Patent Application No. 201910875547.9, filed on Sep. 17, 2019, which is incorporated herein by reference in its entirety.

### TECHNICAL FIELD

The present invention relates to the technical field of biopsy through medical devices, in particular to a rotary oblique-cutting biopsy needle.

### BACKGROUND

In medical diagnosis, tumor patients often need biopsy operations, so that a part of tissue at a lesion location is obtained for pathologic study. Traditional biopsy operations mostly adopt an incision method, but this is gradually being rejected since the incision method has the problem of creating large wounds, high costs, low in safety factors, and the like. In recent years, the biopsy operation is mainly implemented by a biopsy needle percutaneous puncture method, and the biopsy needle percutaneous puncture method has the advantages of creating smaller wounds, less bleeding, higher biopsy success rates, and the like. The practicability, universality, sampling manner, appearance, shape and the like of the biopsy needle have great influence on operation, and therefore, it is particularly important to design a biopsy needle suitable for current biopsy operations.

CN107683114A discloses a method. According to the method, a biopsy needle is designed, and tissue is obtained in a longitudinal cutting manner; and such solution is effective technically, but has some technical defects. Firstly, longitudinal cutting is achieved through longitudinal dislocation of an external needle and an internal needle, and a longitudinal error is easy to occur; secondly, during operation by the method, the external needle and the internal needle need to be inserted firstly at the same time, then the internal needle further penetrates, and finally, the external needle further penetrates to obtain tissue; the operation is complex, and mistakes can occur; finally, the external needle needs to penetrate quickly when the tissue is cut by the external needle; at the moment, the external needle is in contact with the tissue of a human body, the external needle is quickly moved to cause unnecessary injury to the tissue of the human body; and furthermore, when cutting is finished, the position of the needle tip of the external needle is out a tail end of the needle tip of the inner needle, which also results in unnecessary injury to the tissue of the human body.

CN205885466U discloses a method. According to the method, a rotary biopsy needle is designed, tissue is obtained in a rotary cutting manner, therefore, longitudinal errors generated during cutting are reduced effectively, but the method has some techmical defects. Firstly, according to the method, a designed cutting needle sleeve is free from a needle head, and both the puncture process and the cutting process are finished by a cutting needle body; consequently, the cutting needle body with a cutting slot is high in workload, and is easy to break off, and potential safety hazards exist; the needle head of the cutting needle body is an inclined plane of a fixed angle, and therefore, the needle head is not sharp enough, and is difficult to puncture; next, cutting edges of the cutting slot of the cutting needle body designed by the method are both parallel to the axis direction of the needle body, and therefore, rotary cutting is performed in the longitudinal direction at the same time; the rotary cutting operation is independently finished through the cutting needle body; the operation is unreliable due to moveability of the tissue in the human body; thirdly, after the cutting operation is finished through the rotary biopsy needle designed by the method, the cut tissue cannot be isolated for immediate storage, and is easy to lose in the human body, which has great harm to the human body, and can could the biopsy operation to fail; and finally, the rotary biopsy needle designed by the method does not involve a mounting and fixing structure at a tail part, and has no practicality or universality.

### SUMMARY

Based on defects existing in the prior art, to solve the technical problem, the present invention concerns a biopsy needle which is easy to operate by a doctor, does not cause unnecessary injury and secondary injury, can be quickly mounted on an operation trigger, and can cut and store the lesion tissue quickly, safely and effectively.

To solve the above technical problem, the present invention provides a rotary oblique-cutting biopsy needle.

The rotary oblique-cutting biopsy needle includes an internal needle and an external needle, where the internal needle is a solid cylindrical needle, the external needle is a cylindrical needle which is hollow but not run-through, a space between an external needle inner end face and an arc inclination needle tip is solid, and the internal needle and the external needle are in coaxial clearance fit; and an internal needle front end face is in contact with the external needle inner end face to guarantee that an oblique cutting edge and a cutting slot are longitudinally located at the same position, and besides, an internal needle position marking point and an external needle indicator window are also guaranteed to be located at the same longitudinal position.

Preferably, the internal needle includes a transmission gear, a rotary support convex circle, the internal needle position marking point, the oblique cutting edge, a holding tank and the internal needle front end face; the oblique cutting edge is 20 millimeters (mm) in length, and 1 mm in diameter; and the holding tank and the oblique cutting edge are located at the same position, and have equal length.

Preferably, the external needle includes a hexagonal fixing head, the external needle indicator window, the cutting slot, the external needle inner end face and the arc inclination needle tip, and the cutting slot is 20 mm in length, and 1.28 mm in diameter.

Preferably, the oblique cutting edge includes a left knife edge, a right knife edge and a forward inclined plane, where the left knife edge is inclined forwards by 30°, the right knife edge is inclined backwards by 30°, and the forward inclined plane is inclined forwards by 45°.

Preferably, the cutting slot includes a left cutting edge and a right cutting edge, where the left cutting edge and the right cutting edge are both parallel to an axis.

The present invention can have one or more of the following beneficial effects:

(1) when the biopsy needle is used for biopsy operation, a doctor firstly inserts the external needle and the internal needle into the lesion location of a human body at the same time after nesting the external needle and the internal needle, and then the biopsy operation can be finished by only rotating the internal needle by 360°; and besides, in the biopsy operation process, the doctor can observe a state of an internal needle position marking point from an external needle indicator window, so that operation is easy for the doctor, and misoperation due to carelessness is reduced effectively.

(2) The needle tip of the external needle has an arc surface and a horizontal 30° inclined plane which is tangent to the arc surface, the needle tip is sharper due to such design, and the process that the needle punctures the tissue is smoother; and pain from which a patient suffers are relieved effectively, and the operation process is smoother and faster.

(3) The external needle with the needle tip and a cutting slot is used for puncture and supply of cutting environment, and the internal needle with an oblique cutting edge is used for rotary cutting; and therefore, workload of the external needle and the internal needle can be reduced effectively, and operation safety is improved.

(4) The cutting slot of the external needle and the oblique cutting edge of the internal needle have equal length, and the cut tissue is also the same in length as the cutting slot; and unnecessary injury caused by operation is reduced effectively.

(5) The oblique cutting edge of the internal needle includes two knife edges, namely a left knife edge and a right knife edge, where the left knife edge is inclined forwards by 30°, and the right knife edge is inclined backwards by 30°; the rotary cutting process can be guaranteed, cutting is implemented from the longitudinal side firstly, and then performed towards the other side slowly; and therefore, a minimum and sharpest included angle is formed between the knife edges and the cutting slot, and the cutting process is faster and more reliable.

(6) The external needle is fixed during biopsy operation, and the internal needle quickly rotates and moves in the external needle; except for the necessary oblique cutting edge, the internal needle is not in direct contact with the human tissue, and unnecessary injury, caused by the biopsy process, to a human body is reduced effectively.

(7) A holding tank of the internal needle not only can hold pre-cutting tissue for cutting, but also can collect the cut tissue for pathologic study; and the problem that the cut tissue is lost is solved effectively, and operation safety is improved.

(8) A certain space can be formed between a forward inclined plane of the oblique cutting edge of the internal needle and the external needle, and the space further communicates with the holding tank; and therefore, the holding space can be guaranteed, and the situation that rotation of the internal needle is hindered by the cut tissue is avoided.

(9) A transmission gear is arranged at a tail part of the internal needle and used for transmitting torque of a manipulator, so that the internal needle rotates; a rotary support convex circle is arranged at the tail part of the internal needle and can play a role of support and fixation; a hexagonal fixing head is arranged at a tail part of the external needle, so that the external needle does not move or rotate; and therefore, the internal needle and the external needle are higher in universality, and convenient to mount and use.

The above and other features of the invention including various novel details of construction and combinations of parts, and other advantages, will now be more particularly described with reference to the accompanying drawings and pointe d out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In order to facilitate description, the present invention is described in details through the following embodiments and figures. In the accompanying drawings, reference characters refer to the same parts throughout the different views. The drawings are not necessarily to scale; emphasis has instead been placed upon illustrating the principles of the invention. Of the drawings:

FIG. 1 shows a schematic diagram of general assembly.

FIG. 2 shows a schematic diagram of closed assembly.

FIG. 3 shows a schematic diagram of open assembly.

FIG. 4 shows a schematic diagram of tail assembly.

FIG. 5 shows a schematic diagram of tail characteristics of an internal needle.

FIG. 6 shows a schematic diagram of head characteristics of the internal needle.

FIG. 7 shows a schematic diagram of tail characteristics of an external needle.

FIG. 8 shows a schematic diagram of head characteristics of the external needle.

Where: 1 internal needle, 1-1 transmission gear, 1-2 rotary support convex circle, 1-3 internal needle position marking point, 1-4 oblique cutting edge. 1-4-1 left knife edge, 1-4-2 right knife edge, 1-4-3 forward inclined plane, 1-5 holding tank, 1-6 internal needle front end face, 2 external needle, 2-1 hexagonal fixing head, 2-2 external needle indicator window, 2-3 cutting slot, 2-3-1 left cutting edge, 2-3-2 right cutting edge, 2-4 external needle inner end face and 2-5 arc inclination needle tip.

### DETAILED DESCRIPTION

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

In order to make the objective, solution and advantages of a biopsy needle clearer, the following is description of the biopsy needle through embodiments shown in figures. However, it should be understood that the description is exemplary and is not limited to the scope of the biopsy needle. In addition, in the following description, descriptions of conventional structures and technologies are omitted to avoid unnecessary confusing of concepts of the present invention.

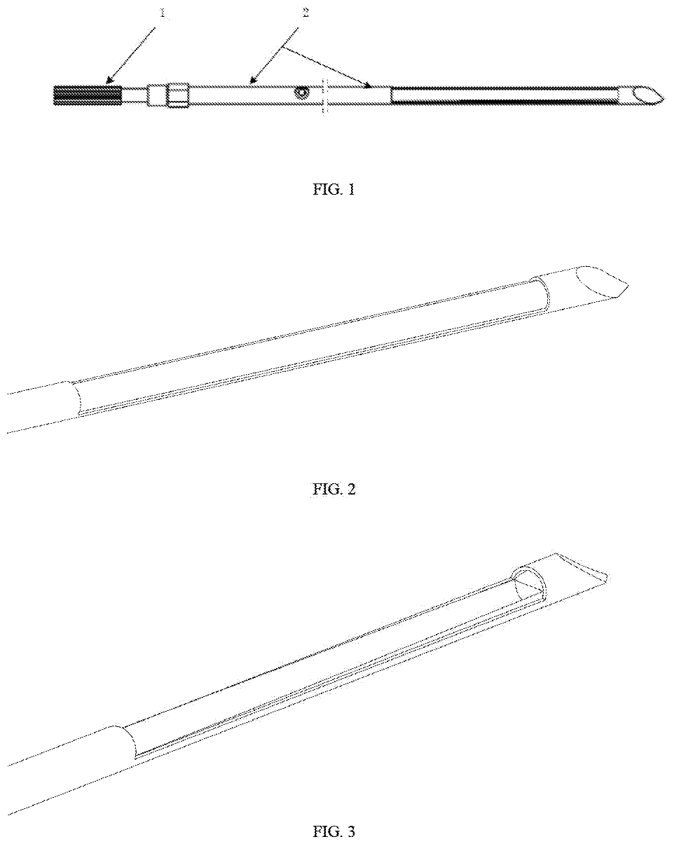

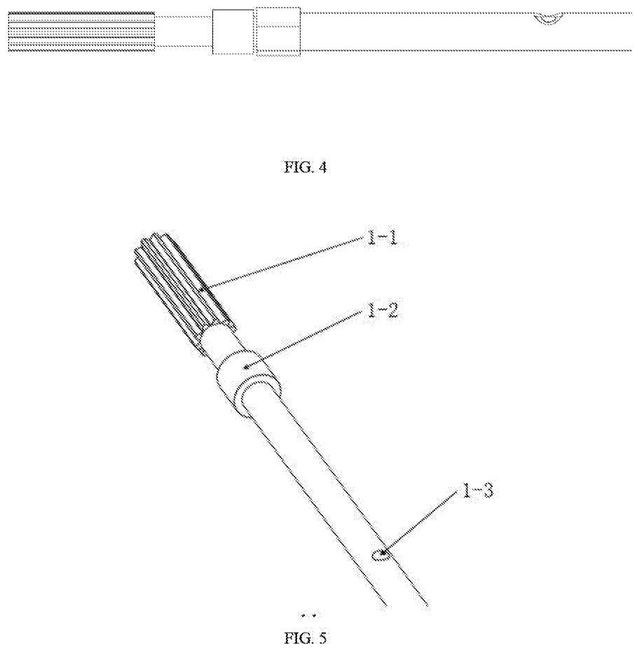

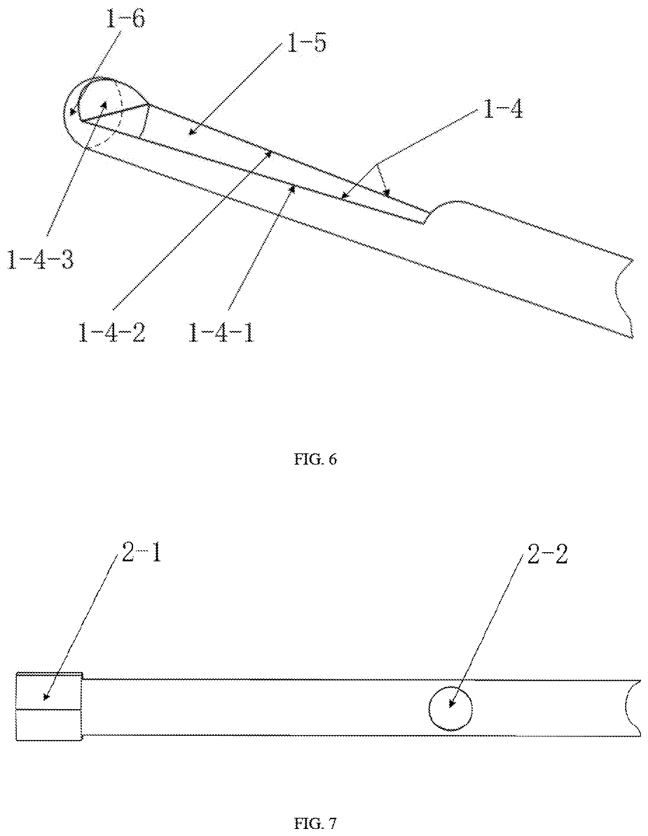

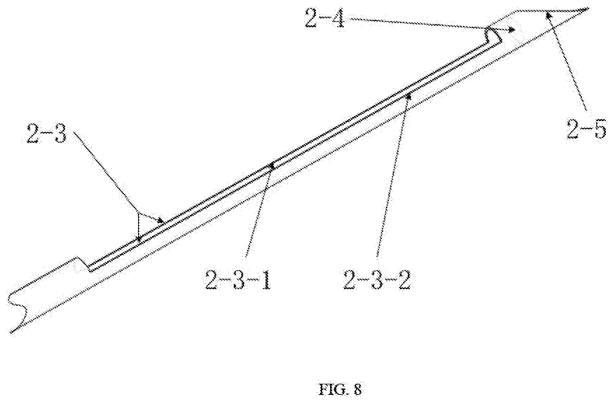

As shown in FIGS. 1-8, embodiments of the present invention adopt the following solution: the biopsy needle includes an internal needle 1 and an external needle 2, where the internal needle 1 is a solid cylindrical needle, the external needle 2 is a cylindrical needle which is hollow but not run-through, a space between an external needle inner end face 2-4 and an arc inclination needle tip 2-5 is solid, and the internal needle 1 and the external needle 2 are in coaxial clearance fit; and an internal needle front end face 1-6 is in contact with the external needle inner end face 2-4 to guarantee that an oblique cutting edge 1-4 and a cutting slot 2-3 are longitudinally located at the same position, and besides, an internal needle position marking point 1-3 and an external needle indicator window 2-2 are longitudinally located at the same position.

Furthermore, the internal needle 1 includes a transmission gear 1-1, a rotary support convex circle 1-2, the internal needle position marking point 1-3, the oblique cutting edge 1-4, a holding tank 1-5 and the internal needle front end face 1-6; the oblique cutting edge 1-4 is 20 mm in length, and 1 mm in diameter; and the holding tank 1-5 and the oblique cutting edge 1-4 are located at the same position, and have equal length.

Furthermore, the external needle 2 includes a hexagonal fixing head 2-1, the external needle indicator window 2-2, the cutting slot 2-3, the external needle inner end face 2-4 and the arc inclination needle tip 2-5, and the cutting slot 2-3 is 20 mm in length, and 1.28 mm in diameter.

Furthermore, the oblique cutting edge 1-4 includes a left knife edge 1-4-1, a right knife edge 1-4-2 and a forward inclined plane 1-4-3, where the left knife edge 1-4-1 is inclined forwards by 30°; and the right knife edge 1-4-2 is inclined backwards by 30°, and the forward inclined plane 1-4-3 is inclined forwards by 45°.

Furthermore, the cutting slot 2-3 includes a left cutting edge 2-3-1 and a right cutting edge 2-3-2, where the left cutting edge 2-3-1 and the right cutting edge 2-3-2 are both parallel to an axis.

The working principles of the embodiments of the present invention are:

before implementation of the biopsy operation, the internal needle 1 needs to be inserted into the external needle 2 until the internal needle front end face 1-6 is in contact with the external needle inner end face 2-4, so that the internal needle 1 and the external needle 2 are in coaxial clearance fit, and are in the closed state shown in FIG. 2; during the operation, the internal needle 1 and the external needle 2 which are in the closed state penetrate into the human body at the same time until the cutting slot 2-3 reaches the lesion location; then, the internal needle 1 is rotated by 180°, so that the assembly of the internal needle 1 and the assembly of the external needle 2 are in the open state shown in FIG. 3; in the open state, the human tissue to be obtained enters the holding tank 1-5 due to pressure, and the internal needle 1 is rotated by 180° in the same direction, so that the internal needle 1 is recovered to the closed state shown in FIG. 2; thus, the tissue to be obtained is cut under the combined action of the oblique cutting edge 1-4 and the cutting slot 2-3, and stored in the holding tank 1-5; after completion of the cutting process, the internal needle 1 and the external needle 2 in the closed state are taken out from the human body at the same time; and the internal needle 1 is rotated by 180° so that the assembly of the internal needle 1 and the assembly of the external needle 2 are located in the open state shown in FIG. 3, then the human tissue stored in the holding tank 1-5 can be taken out, and the biopsy operation is finished.

The above shows and describes the basic principles, main features and advantages of the present invention. It should be understood by those skilled in the art that, the present invention is not limited by the embodiments. The aforementioned embodiments and the description only illustrate the principle of the present invention. Various changes and modifications may be made to the present invention without departing from the spirit and scope of the present invention. Such changes and modifications all fall within the claimed scope of the present invention. The protection scope of the present invention is defined by the appended claims and equivalents thereof.

What is claimed is:

1. A rotary oblique-cutting biopsy needle, comprising an internal needle and an external needle, wherein the internal needle is a solid cylindrical needle, the external needle is a cylindrical needle which is hollow but not run-through, a space between an external needle inner end face and an arc inclination needle tip is solid, and the internal needle and the external needle are in coaxial clearance fit; and an internal needle front end face is in contact with the external needle inner end face to guarantee that an oblique cutting edge and a cutting slot are longitudinally located at the same position, and an internal needle position marking point and an external needle indicator window are longitudinally located at the same position.

2. The rotary oblique-cutting biopsy needle according to claim 1, wherein the internal needle comprises a transmission gear, a rotary support convex circle, the internal needle position marking point, the oblique cutting edge, a holding tank and the internal needle front end face; the oblique cutting edge is 20 mm in length, and 1 mm in diameter; and the holding tank and the oblique cutting edge are located at the same position, and have equal length.

3. The rotary oblique-cutting biopsy needle according to claim 1, wherein the external needle comprises a hexagonal fixing head, an external needle indicator window, the cutting slot, the external needle inner end face and the arc inclination needle tip, and the cutting slot is 20 mm in length, and 1.28 mm in diameter.

4. The rotary oblique-cutting biopsy needle according to claim 1, wherein the oblique cutting edge comprises a left knife edge, a right knife edge and a forward inclined plane, and the left knife edge is inclined forwards by 30°; and the right knife edge is inclined backwards by 30°, and the forward inclined plane is inclined forwards by 45°.

5. The rotary oblique-cutting biopsy needle according to claim 1, wherein the cutting slot comprises a left cutting edge and a right cutting edge, and the left cutting edge and the right cutting edge are both parallel to an axis.
